(19) United States
(12) Patent Application Publication
(10) Pub. No.: US 2025/0370127 A1
(43) Pub. Date: Dec. 4, 2025

(54) ULTRASOUND SYSTEMS AND DEVICES WITH IMPROVED ACOUSTIC PROPERTIES

(71) Applicant: Exo Imaging, Inc., Santa Clara, CA (US)

(72) Inventors: Killian SULLIVAN, Menlo Park, CA (US); Huseyin Kagan OGUZ, San Jose, CA (US); Anthony BROCK, Half Moon Bay, CA (US); Michael CONROY, Alameda, CA (US); Thomas TARTER, San Jose, CA (US); Drake GUENTHER, Hillsborough, CA (US); Brian BIRCUMSHAW, Oakland, CA (US)

(21) Appl. No.: 19/234,955

(22) Filed: Jun. 11, 2025

Related U.S. Application Data
(63) Continuation of application No. 17/507,615, filed on Oct. 21, 2021, now abandoned.

(51) Int. Cl. G01S 15/89; A61B 8/00; H05K 7/20

(52) U.S. Cl. CPC G01S 15/8915; H05K 7/2039; A61B 8/546

(57) ABSTRACT

Acoustic imaging artifacts produced by imaging systems and devices can be reduced or eliminated by including a multisink medium in the systems or devices. The multisink material can be disposed between an acoustically reflective component of the imaging system or device and an ultrasound transducer of the imaging system or device. Inclusion of a multisink medium that is thermally conductive and acoustically non-conductive can reduce acoustic imaging artifacts while maintaining or improving heat management within imaging systems and devices.

### CROSS-REFERENCE TO RELATED APPLICATIONS

This application is a continuation of U.S. patent application Ser. No. 17/507,615, filed Oct. 21, 2021, the entirety of which is incorporated herein by reference.

### BACKGROUND

Ultrasound imaging is a common technique for medical and non-destructive testing. Ultrasound systems and devices typically comprise an ultrasonic transducer capable of producing and transmitting acoustic energy along a transmission axis into an acoustically heterogeneous target substance and detecting acoustic energy reflected from the target substance to create an image along an axis of acoustic wave transmission. Partial or complete reflection of the transmitted acoustic energy can occur when a transmitted acoustic energy wave encounters an interface between a first portion of the target substances having a first acoustic impedance (Z₁) and a second portion of the target substance having a second acoustic impedance (Z₂). A reflection coefficient (R), which can be used to determine the amplitude of the reflected acoustic energy wave can be calculated using Equation (1):

$$R = \frac{{Z}_{2} - {Z}_{1}}{{Z}_{2} + {Z}_{1}} \qquad Equation ( 1 )$$

However, performance of ultrasound systems and devices can be adversely impacted by the sources of acoustic noise within the immediate environment of an ultrasound probe transducer and/or a target substance of an ultrasound scan. Reverberation (reverb) artifacts can arise when an ultrasonic energy wave reflects between two or more parallel acoustic reflectors. In some cases, the deleterious effects of reverb artifacts in a target substance comprising parallel acoustic reflectors can be lessened by changing the angle at which the acoustic wave is transmitted to the target substance. However, such strategies can only be employed when it is possible to change the angle of the ultrasound transducer relative to the parallel acoustic reflectors. There exists a long-felt and unresolved need to address situations in which reverberation artifacts arise from acoustic noise sources for which the angle of incidence of transmitted ultrasonic waves is not easily changed.

### SUMMARY

Ultrasound imaging artifacts arising from sources outside of an ultrasound scan target (e.g., a target substance) can present unique challenges, for example, because adjustments to the design of an ultrasound device or system to address such hindrances can involve substantial performance tradeoffs and design compromises. Described herein are systems, devices, and methods that can improve ultrasound scan performance with respect to artifacts arising from acoustic reverberation within an ultrasound scanning device. In various aspects, systems, devices, and methods can reduce the impact of acoustic reverberation on ultrasound scan quality while also surmounting competing design constraints related to heat management, cost, and/or probe geometry constraints. For instance, some embodiments described herein include a multisink medium (e.g., an injectable multisink medium 400), which can improve the acoustical isolation of ultrasound transducers of an ultrasound system or device (e.g., from acoustically reflective components of the ultrasound system or device) while simultaneously improving heat conduction within a probe of the system or device.

In various aspects, an imaging device comprises: an integrated circuit substrate; a multisink medium in contact with the integrated circuit substrate; and one or more microelectromechanical (MEMs) ultrasound transducers coupled to the integrated circuit substrate. In some cases, the imaging device further comprising a heatsink. In some cases, the heatsink comprises a metal. In some cases, the metal is aluminum. In some cases, the multisink medium is in contact with the heatsink. In some cases, the multisink medium is disposed at least partially between the one or more MEMs transducers and the heatsink. In some cases, the device further comprises a housing coupled to the integrated circuit substrate. In some cases, the multisink medium is in contact with the housing. In some cases, the multisink medium is disposed at least partially between the one or more MEMs transducers and the housing. In some cases, the multisink medium is injectable. In some cases, the multisink medium has a flow rate of at least 29 g/min. In some cases, the multisink medium has a flow rate of at least 40 g/min. In some cases, the multisink medium has a thermal conductivity of at least 1.5 Watts per meter-Kelvin (W/mK). In some cases, the multisink medium has a thermal conductivity of at least 3.7 Watts per meter-Kelvin (W/mK). In some cases, the multisink medium has a thermal conductivity of at least 6.4 Watts per meter-Kelvin (W/mK). In some cases, the multisink medium has a thickness of at least 0.5 mm. In some cases, the multisink medium has a thickness of at least 1.0 mm. In some cases, the multisink medium has a thickness of at least 1.5 mm. In some cases, the imaging device further comprises a backing material. In some cases, the backing material comprises a backing laminate.

In further aspects, a method of fabricating an imaging device comprises the steps of: forming an internal cavity by coupling a first component of the imaging device to an integrated circuit substrate; coupling one or more microelectromechanical (MEMs) ultrasound transducers to the integrated circuit substrate; and injecting a multisink medium into the internal cavity. In some cases, the first component comprises an acoustically reflective material. In some cases, the first component is coupled directly to the integrated circuit substrate. In some cases, the first component is a heatsink. In some cases, the heatsink comprises a metal. In some cases, the multisink medium is in contact with the heatsink. In some cases, the first component comprises a housing. In some cases, the multisink medium is in contact with the housing. In some cases, the multisink medium is disposed at least partially between the one or more MEMs transducers and the first component. In some cases, the multisink medium is injectable. In some cases, the multisink medium has a flow rate of at least 29 g/min. In some cases, the multisink medium has a flow rate of at least 40 g/min. In some cases, the multisink medium has a thermal conductivity of at least 1.5 Watts per meter-Kelvin (W/mK). In some cases, the multisink medium has a thermal conductivity of at least 3.7 Watts per meter-Kelvin (W/mK). In some cases, the multisink medium has a thermal conductivity of at least 6.4 Watts per meter-Kelvin (W/mK). In some cases, the multisink medium has a thickness of at least 0.5 mm after injection. In some cases, the multisink medium has a thickness of at least 1.0 mm after injection. In some cases, the multisink medium has a thickness of at least 1.5 mm after injection. In some cases, the method further comprises a step of coupling a backing material to the integrated circuit substrate. In some cases, the backing material comprises a backing laminate. In some cases, the backing material is disposed at least partially between the first component and the one or more MEMs ultrasound transducers.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a block diagram of an imaging device that can comprise a multisink medium, in accordance with embodiments;

FIG. 2 shows a schematic diagram of an imaging device that can comprise a multisink medium, in accordance with embodiments;

FIG. 3 shows a schematic diagram of internal components of an imaging device, in accordance with embodiments;

FIG. 4 shows a schematic diagram of an alternate configuration of internal components of an imaging device, in accordance with embodiments;

FIG. 5 shows a schematic diagram of a second alternate configuration of internal components of an imaging device, in accordance with embodiments;

FIG. 6 shows a schematic diagram of a third alternate configuration of internal components of an imaging device, in accordance with embodiments;

FIG. 7A shows an ultrasound scan of a test target substance using aluminum backing and no multisink medium, in accordance with embodiments;

FIG. 7B shows an ultrasound scan of the test target substance of FIG. 7A with identical scan settings using multisink medium, in accordance with embodiments;

FIG. 7C shows an ultrasound scan of the test target substance of FIG. 7A with identical scan settings using air backing and no multisink medium, in accordance with embodiments;

FIG. 8A shows an ultrasound scan of a test target substance using multisink medium having 0.5 millimeters thickness, in accordance with embodiments;

FIG. 8B shows an ultrasound scan of the test target substance of FIG. 8A with identical scan settings using multisink medium having 1.0 millimeters thickness, in accordance with embodiments;

FIG. 8C shows an ultrasound scan of the test target substance of FIG. 8A with identical scan settings using air backing and no multisink medium, in accordance with embodiments;

FIG. 9A shows an ultrasound scan of a test target substance using multisink medium 400 having 0.5 millimeters thickness, in accordance with embodiments;

FIG. 9B shows an ultrasound scan of the test target substance of FIG. 9A with identical scan settings using multisink medium having 1.0 millimeters thickness, in accordance with embodiments; and

FIG. 9C shows an ultrasound scan of the test target substance of FIG. 9A with identical scan settings using air backing and no multisink medium, in accordance with embodiments.

FIG. 10 shows a block diagram of a computing system, in accordance with embodiments.

### DETAILED DESCRIPTION

Disclosed herein are systems, devices, and methods for improved acoustic and heat management in ultrasound imaging applications. The geometry and materials comprising ultrasound imaging systems and devices can give rise to multiple, potentially competing design constraints, including heat management and the reduction of acoustic artifacts. Heat transfer away from temperature-sensitive electronic components of an imaging system or device (e.g., a probe head of an ultrasound imaging system or device) is typically handled in existing instruments by incorporation of thermally conductive metal heatsinks, which act as a conduit for heat produced by the electronic components. Unfortunately, metal heatsinks can create acoustic artifacts, including reverberation artifacts, which can result from ultrasound energy (e.g., ultrasound waveforms or patterns) produced by a transducer of an ultrasound device (e.g., a transducer of an ultrasound probe head) traveling backward through the body of the device, reflecting off of the metal heatsink (and/or other components that can reflect ultrasound energy), and returning to the detection transducer of the device. Meanwhile, many acoustically insulating materials such as air, which can be present in internal cavities or spaces of an imaging system or device, do not conduct thermal energy efficiently, reducing the overall efficiency of conduction of heat away from heat-sensitive internal components of the imaging system or device, such as processors and integrated circuits in an ultrasound probe head. As described herein, a multisink medium 400, which can comprise a substance (e.g., a silicone-based paste or putty comprising ceramic particles) capable of conducting thermal energy efficiently and simultaneously absorbing or dissipating acoustic energy (e.g., ultrasound energy), can be incorporated into imaging systems and devices to improve heat management and acoustic isolation of imaging transducers at the same time. In many cases, multisink medium 400, which can be used in an injectable or moldable paste or putty form, can easily fill irregularly shaped cavities, gaps, or spaces in an imaging system or device (e.g., in an ultrasound probe head), providing a greater cross-sectional area for heat flux (e.g., from an electrical component to a metal heatsink or housing, which, in addition to potential for generating acoustic artifacts, may not be easily fabricated to fill such cavities, gaps, and spaces) and allowing more closely and/or complete contouring (e.g., acoustic isolation) of acoustically reflective materials.

As described herein, an imaging system or device 100 can include hardware and/or software configured to transmit and/or receive ultrasonic energy (e.g., in the form of ultrasonic waveforms or groups or patterns of ultrasonic waveforms). In many cases, an image of all or a portion of a target substance (e.g., a target tissue) can be produced by processing and/or analysis of ultrasonic energy (e.g., ultrasonic waveforms or patterns of ultrasonic waveforms) by an imaging device or system, for instance, an ultrasound transducer of an imaging device or system described herein. In some cases, embodiments of the present disclosure can relate to imaging devices and systems, for example, non-intrusive ultrasonic imaging devices and systems comprising microelectromechanical system (MEMs) ultrasound transducers. In some cases, an ultrasound transducer can comprise a MUT (e.g., a transducer unit (e.g., a “pixel”) with a single diaphragm or membrane). In some cases, an ultrasound system or device comprises a transducer element, which can comprise a plurality of transducer units grouped to function together as one. In some cases, an ultrasound transducer can convert received ultrasound energy (e.g., a received ultrasound waveform or pattern) into an electrical signal or pattern. In some cases, an ultrasound device or system can be configured to convert an electrical signal or pattern produced by a transducer from a received ultrasound waveform or pattern into an image of all or a portion of a target substance.

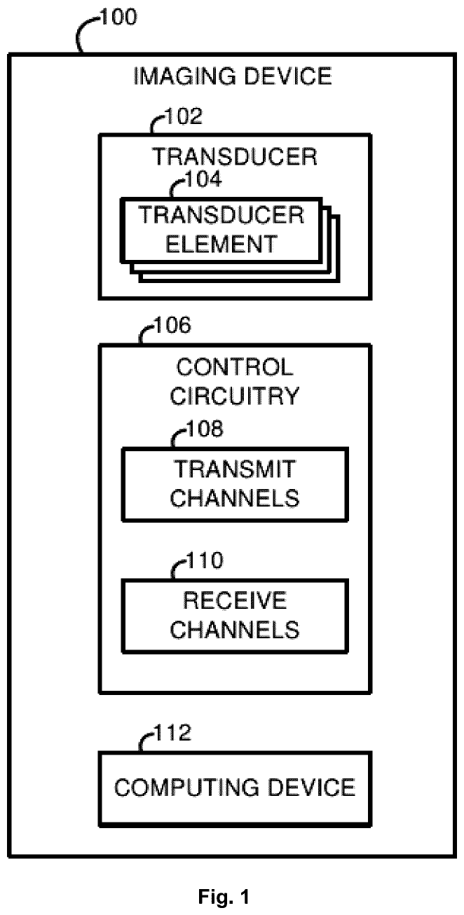

As shown in FIG. 1, an imaging system or device 100 can comprise a transducer 102. A transducer 102 of an imaging system or device 100 can comprise one or more transducer elements 104 (e.g., a plurality of transducer elements, for example, arranged in an array). In some cases, a transducer 102 of an imaging system or device 100 can comprise a plurality of transducer elements 104. A transducer element 104 can comprise a plurality of transducer units, which may each comprise a piezoelectric micromachined ultrasound transducer (pMUT) or a capacitive micromachined ultrasound transducer (cMUT). A pMUT or cMUT can operate based on photo-acoustic or ultrasonic principles, e.g., in the imaging of a target substance. A transducer element 104 or portion thereof (e.g., a transducer unit) can be used to generate an ultrasonic pressure wave (e.g., ultrasound energy), which propagate through a target substance, which can comprise biological tissue (e.g., comprising bones, blood flow, and/or organ(s) of a human or animal) and/or other substances or masses. A transducer element 104 or portion thereof (e.g., a transducer unit) can be used to receive ultrasonic energy (e.g., that has been reflected from a portion of the target substance). In many cases, a transducer element 104 or portion thereof can be configured to convert received ultrasonic energy to an electrical signal. In some cases, an imaging system or device 100 can transmit a signal (e.g., an ultrasonic waveform or pattern) into the target substance (e.g., the body or portion thereof) and receive a reflected signal (e.g., an ultrasonic waveform or pattern) from the target substance (e.g., the body or portion thereof). In some cases, an imaging system or device can be configured to simultaneously transmit and receive ultrasonic energy (e.g., comprising one or more ultrasound waveforms or patterns).

A transducer (or plurality of transducers), such as a pMUT or cMUT, may be efficiently formed on a substrate 260, e.g., in methods utilizing semiconductor wafer manufacturing processes. Compared to conventional transducers (e.g., traditional bulk piezoelectric (PZT) transducers), pMUT transducer elements and pixels can be built on semiconductor substrates 260 (e.g., integrated circuit substrates), which can be less bulky, less expensive to manufacture, less complicated, and can have higher performing electronic/transducer interconnections than traditional PZT transducer substrates. In many cases, imaging systems and devices 100 comprising pMUTs can allow greater flexibility in operational frequency and can generate higher quality images.

In some cases, a substrate 260 (e.g., an integrated circuit substrate) can comprise a semiconductor wafer. In some cases, a semiconductor wafer can be 6 inches, 8 inches, 12 inches, 6 to 8 inches, 8 to 12 inches, 6 to 12 inches, less than 6 inches, or greater than 12 inches in length. In some cases, a semiconductor wafer can be manufactured by forming one or more silicon dioxide (SiO₂) layers on a silicon substrate. Further processing in the manufacture of a semiconductor wafer can include addition (e.g., comprising processes of deposition or etching) of metal layers or paths, e.g., to serve as interconnects and bond pads for electronic components to be coupled to the semiconductor wafer integrated circuit substrate 260. In some cases, cavities can be etched into the integrated circuit substrate 260.

An imaging system or device 100 can comprise an application specific integrated circuit (ASIC), which can comprise electronics for operation of transducers, formation of transmitted ultrasonic waveforms or patterns, and/or processing of electronic signals produced by transducers upon receiving (e.g., reflected) ultrasonic energy (e.g., from a target substance or portion thereof). In some cases, an ASIC can comprise one or more transmit drivers, sensing circuitry (e.g., to process electrical energy corresponding to received ultrasound energy, which may have been received by a transducer after reflecting back from an object or substance to be imaged (e.g., echo signals)), and/or other processing circuitry to control other operations associated with the function of the imaging system or device 100. In some cases, an ASIC can be formed on a substrate 260 (e.g., a semiconductor wafer, such as a semiconductor wafer integrated circuit substrate). In some cases, an ASIC can be located (e.g., positioned within the imaging system or device 100) in close proximity to transducer (e.g., pMUT or cMUT) elements or pixels of the imaging system or device 100, for instance, to reduce parasitic loss. For example, an ASIC can be located 50 micrometers or less from a transducer (e.g., an array of transducer elements). An ASIC can be directly coupled to a substrate 260 (e.g., semiconductor wafer integrated circuit substrate) of an imaging system or device 100. In some cases, an ASIC is directly coupled to the same integrated circuit substrate 260 (e.g., semiconductor wafer substrate) as a transducer of the imaging system or device 100 is directly coupled to. For example, a transducer of the imaging system or device 100 can be coupled to an integrated circuit substrate 260 on which the ASIC is manufactured, for instance using low temperature piezo material sputtering and/or other low temperature processing compatible with ASICs. In some cases, an ASIC is indirectly coupled to an integrated circuit substrate 260 (e.g., semiconductor wafer substrate) that is directly coupled to a transducer of the imaging system or device 100, 200, 250 (e.g., via stacked wafer-to-wafer interconnection). For example, an ASIC may be directly coupled to a first integrated circuit substrate 260 that is indirectly coupled to a transducer (e.g., wherein the first integrated circuit substrate is directly coupled to a second integrated circuit substrate 260, which is directly coupled to the transducer). In some cases, an ASIC (or integrated circuit substrate 260 coupled to the ASIC) may be spatially separated from a transducer element or array (or semiconductor wafer substrate 260 coupled to a transducer element or array) by less than 100 micrometers. In some cases, an ASIC can have a similar or identical footprint relative to a pMUT transducer (e.g., comprising pMUT elements). An ASIC can be coupled to a transducer via interconnects.

One or more transducers 104 (e.g., one or more transducer elements or pixels) of an imaging system or device 100, 200, 250 can be configured to transmit or receive ultrasonic signals (e.g., ultrasonic energy, e.g., in the form of ultrasonic pressure waves or patterns of waveforms) at a specific frequency and bandwidth (or within a range of frequencies and with a range of bandwidths). In some cases, a transducer 104 (or array of transducer elements) can be configured to transmit or receive signals (e.g., ultrasonic energy, e.g., in the form of ultrasonic pressure waves or patterns of waveforms) at a plurality of frequencies and bandwidths, for example, comprising a first center frequency (and a first bandwidth) and one or more additional center frequencies (having one or more corresponding additional bandwidths). In some cases, a transducer 104 (e.g., a transducer array, element, or pixel) can emit (e.g., transmit) or receive ultrasonic signals having a center frequency from 0.1 megahertz (MHz) to 100 MHZ (e.g., from 0.1 MHz to 1.8 MHz, from 1.8 MHz to 5.1 MHz, or higher than 5.1 MHZ). In some cases, an ultrasonic signal (e.g., an ultrasonic waveform, pattern, or pressure wave) can be generated by employing one or more transmit channels 108 to drive one or more transducers of a transducer array 102 (e.g., or group of transducer elements 104 or pixels) with a voltage pulse at a frequency to which the one or more transducers are response. In many cases, this can cause an ultrasonic waveform to be emitted (e.g., transmitted) from the transducer elements 104 toward a target substance, for example, when imaging of the target substance or a portion thereof is desired. In some cases, an ultrasonic waveform can include one or more ultrasonic pressure waves transmitted from one or more corresponding transducer elements of the imaging device, for example, wherein the pressure waves are transmitted simultaneously or substantially simultaneously. Ultrasonic energy (e.g., transmitted toward a target substance by a transducer, e.g., in the form of a waveform or pattern, as described herein) can travel toward, into, and/or through the target substance. In many cases, all or a portion of the transmitted ultrasonic energy can be reflected back to the transducer 102. In many cases, ultrasonic energy reflected back to the transducer 102, e.g., from a target substance or portion thereof, can be received by the transducer 102. Ultrasonic energy received by the transducer 102 (e.g., after reflecting back to the transducer 102) can be converted into electrical energy (e.g., an electrical signal) through a piezoelectric effect at the transducer 102. One or more receive channels 110 can collect electrical energy produced by the transducer 102 (e.g., as a result of converting ultrasonic energy to an electrical signal). In some cases, the one or more receive channels 110 can process the electrical energy. In some cases, electrical energy produced by a transducer 102 (e.g., and collected by a receive channel 110) can be transmitted to a computing system or device 112, e.g., for processing, which may include generation of an image that can be displayed.

An imaging system or device 100, 200, 250 can comprise control circuitry 106, which can comprise a controller. Control circuitry 106 of an imaging system or device 100, 200, 250 can be configured to control (e.g., operate) one or more transducer elements 104 or transducer units of the imaging system or device 100, 200, 250. In some cases, control circuitry 106 can be configured to operate transducer elements 104 to receive ultrasonic energy (e.g., comprising ultrasonic pressure waves) reflected back to the transducer elements 104, and to generate electrical signals based on the received ultrasonic energy. In some cases, control circuitry 106 of an imaging system or device 100, 200, 250 can comprise one or more transmit channels 108 and one or more receive channels 110. In some cases, control circuitry can comprise beamforming circuitry. In some cases, control circuitry 106 comprises an application specific integrated circuit (ASIC), a field programmable gate array (FPGA), a system-on-a-chip, a processor, a memory (e.g., a non-transitory memory and/or a transitory memory), a voltage source, a current source, one or more amplifiers (e.g., one or more operational amplifiers), one or more digital-to-analog converters, and or one or more analog-to-digital converters. In some cases, a transducer 104 can be coupled to one or more transmit driver circuits (e.g., of a transmit channel described herein) and/or a low-noise amplifier (e.g., of a receive channel). In some cases, a transmit channel can include transmit drivers. In some cases, a receive channel can comprise one or more low noise amplifiers. In some cases, the transmit and receive channels can each include multiplexing and address control circuitry, e.g., to enable specific transducer elements and sets of transducer elements to be activated, deactivated, or put in low power mode.

An imaging system or device 100, 200, 250 can comprise a computing system 112 (e.g., a computing device 112), as described herein. In some cases, a computing system or device can comprise a processor, a memory (e.g., a non-transitory memory and/or a transitory memory), communication circuitry (e.g., wireless or wired communication ports and/or communication modules), a battery, and/or a display. A computing system or device 112 can be integrated with (e.g., coupled to) the control circuitry 106, and/or one or more transducer elements, pixels, or arrays 102. In some cases, a computing system or device 112 can comprise a plurality of components (e.g., components described herein, such as the control circuitry, transducers, and/or multisink medium 400) coupled to a single substrate 260 (e.g., chip), for example, as a single system on a chip (SoC), or disposed within the same housing. In some cases, a computing system or device 112 of an imaging system or device 100, 200, 250 can be coupled to but physically separate from (e.g., not located on the same chip or in the same housing as) the control circuitry, transducers, and/or multisink medium 400.

In some cases, an imaging system or device can be configured to transmit firings (e.g., transmissions of ultrasonic waveforms from a transducer element toward, into, or through a target substance) in a manner that controls power dissipation without exceeding temperature limits of the imaging device, while simultaneously maintaining required image quality. In some cases, the number or pattern of transmit and/or receive channels can be dynamically controlled (e.g., by control circuitry 106), for example to reduce power consumption and/or reduce a risk of overheating in the device. In some cases, the number of transmit channels 108 and/or the number of receive channels 110 of an imaging system or device 100, 200, 250 in operation is constant through the operation of the imaging system or device 100, 200, 250. In some cases, the number of transmit channels in operation is not constant through the operation of the imaging system or device 100, 200, 250. For example, an imaging system having transducer elements 104 arranged in a two-dimensional spatial array with N columns and M rows (e.g., arranged in a orthogonal rows and columns or arranged in asymmetric (or staggered) rectilinear arrays) may employ as many as N transmit channels and/or receive channels, as many as M transmit channels and/or receive channels, as many as N×M transmit channels and/or receive channels throughout or at any point during operation of the transducer array of the imaging system or device 100, 200, 250. For example, a subset of the transducer elements 104 may be used for a given ultrasound scan or portion of an ultrasound scan. In some cases, a transducer element may be coupled to both a transmit channel 108 and a receive channel 110. For example, a transducer element 104 may be configured to create and transmit an ultrasound pulse and then detect the echo of that pulse, e.g., by converting the reflected ultrasonic energy into electrical energy. In some cases, a plurality of transducers 104 can be coupled to the same one or more transmit channels 108 and/or the same one or more receive channels 110. In some cases, each of the transducers 104 can be coupled to a different transmit channel 108 and/or a different receive channel 110. In some cases, any or all of the transducer elements 104 may be coupled to either a transmit channel 108 or a receive channel 110 but not both.

As described herein, an imaging system or device 100, 200, 250 can comprise multisink medium 400 (e.g., in systems and devices 100 having or lacking dynamic control of transmit and/or receive channels), for instance to improve heat management and/or reduce imaging artifacts (e.g., reverberation artifacts resulting from acoustically reflective components of the imaging system or device 100, 200, 250).

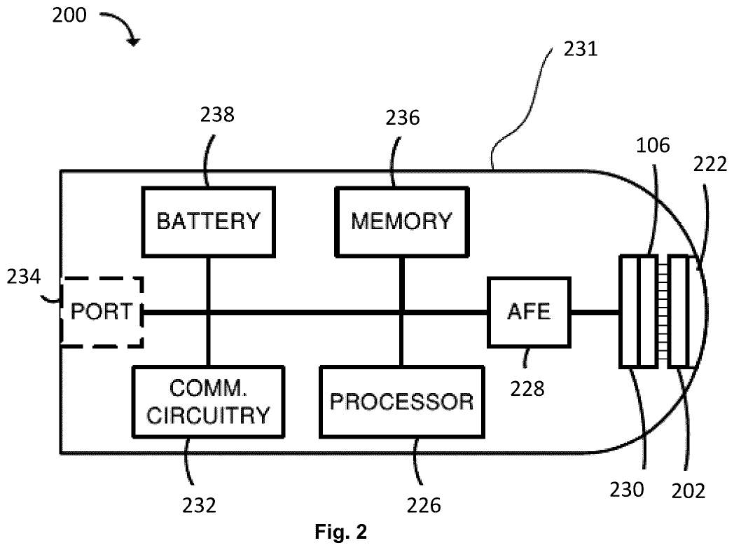

FIG. 2 is a schematic diagram of an imaging device 100, 200, 250 with selectively adjustable features, according to some embodiments. The imaging device 200, 250 may be similar to imaging device 100 of FIG. 1, by way of example only. Imaging device 100, 200, 250 may comprise an ultrasonic medical probe.

As shown in FIG. 2, an imaging system or device 100, 200, 250 or portion thereof (e.g., a probe of an imaging system or device 100, 200, 250) can comprise a housing 231 (e.g., a handheld casing), e.g., which can house transducers 202 and associated electronics. In some cases, an imaging system or device 100, 200, 250 or portion thereof (e.g., a probe of an imaging system or device 100, 200, 250) can comprise a battery 238, e.g., to power one or more components of the imaging system or device 100, 200, 250 or portion thereof, e.g., as shown in FIG. 2. In some cases, an imaging system or device 100, 200, 250 or portion thereof, e.g., as shown in FIG. 2, can comprise a portable imaging device capable of two-dimensional (2D) and/or three-dimensional (3D) imaging using pMUT transducer elements arranged in a 2D array, optionally built on (e.g., coupled to) a substrate 260 (e.g., a silicon wafer integrated circuit wafer). In some cases, one or more transducers (e.g., a transducer array) can be coupled (e.g., directly or indirectly) to an application specific integrated circuit (ASIC) 106, which can aid in controlling parameters of transducer operation, e.g., as described herein. Although not shown explicitly in FIG. 2, an imaging system or device 100, 200, 250 or portion thereof (e.g., as shown in FIG. 2) can comprise a multisink, which can be disposed within the housing, for example, between a transducer (and/or an ASIC, and/or a substrate 260, such as an integrated circuit substrate 260) and one or more of a heatsink, a housing, or one or more additional components shown in FIG. 2.

As shown in FIG. 2, an imaging device 100, 200, 250 can comprise one or more transducers 202. In some cases, the one or more transducers 202 can comprise one or more arrays of transducer elements, for example, wherein the transducers 202 may be configured to transmit and/or receive ultrasonic energy (e.g., ultrasonic pressure waves).

In some cases, imaging device 100, 200, 250 can comprise a coating layer 222, e.g., to serve as an impedance matching interface between the transducers 202 and a target substance (e.g., the human body or other mass or tissue through which ultrasonic energy is to be transmitted by the imaging device 100, 200, 250). In some cases, coating layer 222 can be a lens or can function as a lens, e.g., when designed with a curvature consistent with a desired focal length. In some cases, a user may apply a gel to the surface of a target substance (e.g., to the skin of a living body) prior to contacting coating layer 222 to the surface of the target substance, e.g., to improve impedance matching at the interface of the coating layer 222 and the surface of the target substance. In some cases, matching impedance as described herein can improve conduction of acoustic energy at the interface of the coating layer 222 and the surface of the target substance (e.g., either during transmission into the target substance or during receiving of reflected acoustic energy from the target substance) and can reduce loss of (e.g., amplitude) of the acoustic energy. Coating layer 222 may be a flat layer, e.g., to maximize transmission of acoustic signals from (e.g., a flat array of) transducers 202 to the body and vice versa. Coating layer 222 can have a thickness equal to one quarter (e.g., 25%) of the wavelength of the acoustic pressure wave to be generated at the transducer 202, in some embodiments.

Imaging device 100, 200, 250 can comprise control circuitry 106. Control circuitry 106 can comprise one or more processors, e.g., for controlling one or more transducers 202. In some cases, control circuitry 106 comprises an application-specific integrated circuit (ASIC) or ASIC chip. In some cases, an ASIC or ASIC chip can comprise one or more processors. Control circuitry 106 can be coupled to one or more transducers 102, e.g., by way of bumps, for example in a stacked configuration, in some embodiments. In some cases, control circuitry can be configured to (e.g., selectively) operate and/or adjust the operation of transmit channels 108 and/or receive channels 110, for example based on a desire to test pixels for defects and/or to change a mode of scanning or otherwise adjust the operation of the transducers 102. In some cases, imaging device 100, 200, 250 can comprise one or more processors 226, e.g., for controlling one or more components of imaging device 100, 200, 250. One or more processors 226 can be configured to control operation of one or more transducer elements (e.g., in coordination with or independently of control circuitry 106), to process electrical signals (e.g., based on reflected ultrasonic energy received by transducer elements), and/or to generate signals (e.g., to cause a restoration of an image of a target substance or portion thereof being imaged by one or more processors of a computing device, such as computing device 112). In some cases, one or more processors 226 can be configured to perform other processing functions associated with imaging device 100, 200, 250. Processor(s) 226 can be embodied as any type of processor(s). For example, one or more processors 226 can be embodied as single or multi-core processor(s), single or multi-socket processor(s), digital signal processor(s), graphics processor(s), neural network computer engine(s), image processor(s), microcontroller(s), field programmable gate array(s) (FPGAs), or other processor/controlling circuit(s).

Imaging device 100, 200, 250 can comprise circuit(s) 228, which can comprise Analog Front End (AFE) (e.g., for processing/conditioning signals). In some cases, analog front end 228 can be embodied as any circuit or circuits configured to interface with the control circuitry 106 and other components of the imaging device, such as processor 226. For example, analog front end 228 can include, e.g., one or more digital-to-analog converters, one or more analog-to-digital converters, and/or one or more amplifiers.

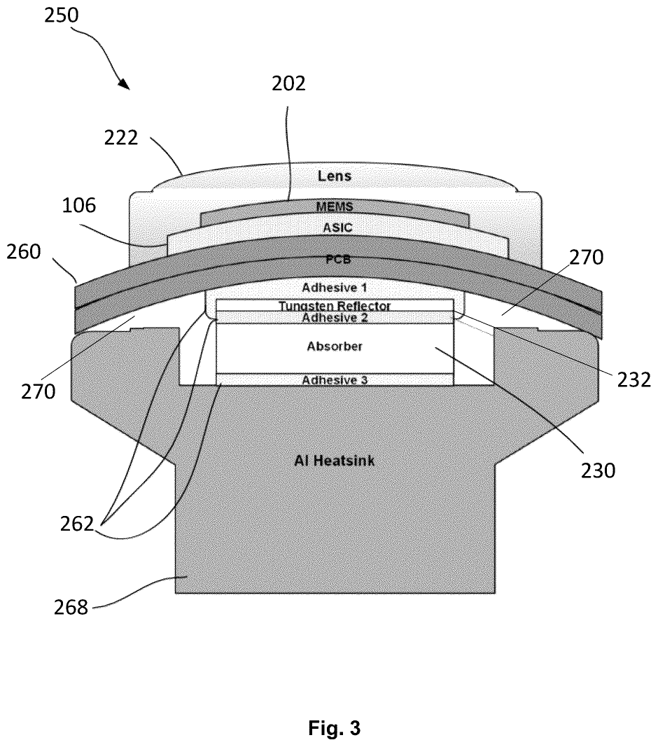

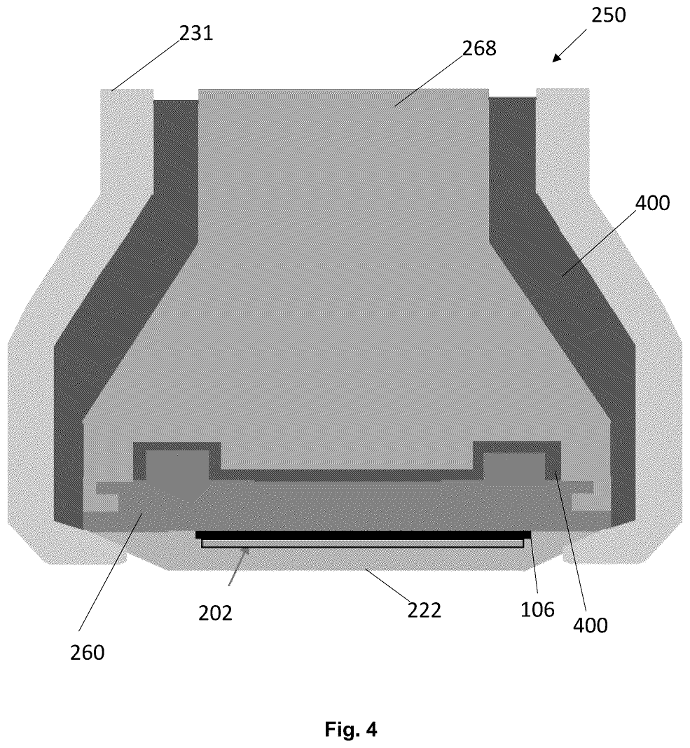

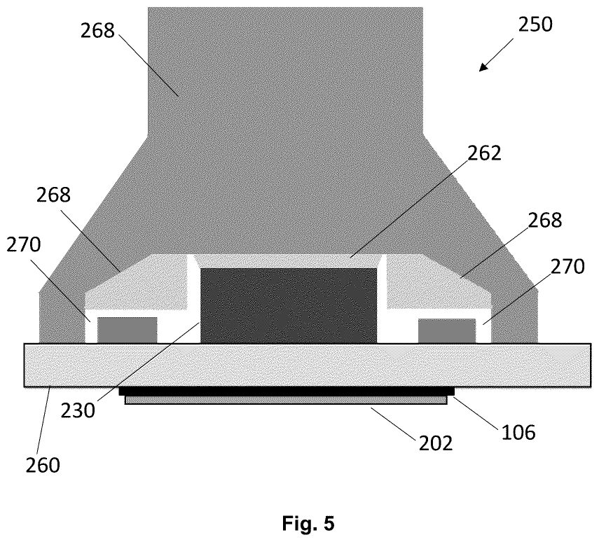

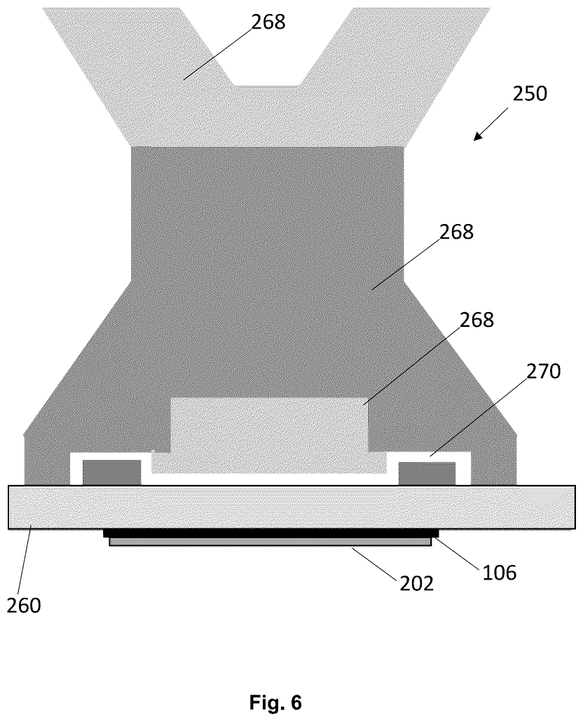

Imaging device 100, 200, 250 can comprise a multisink medium 400 and/or an acoustic absorber layer 230 (e.g., for absorbing acoustic energy generated by transducers 202 and propagated toward circuits 228), for instance as illustrated in FIG. 3, FIG. 4, FIG. 5, and FIG. 6. A multisink medium 400 (and, in some cases, an acoustic absorber layer) can absorb ultrasonic energy emitted in a reverse direction (e.g., emitted by transducers 202 in a direction away from coating layer 222), which may otherwise be reflected and interfere with the quality of the image (e.g., through the generation of artifact(s), such as reverberation artifacts). For example, transducer(s) 202 (e.g., which may be mounted on a substrate 260) can be in contact with a multisink medium 400 (e.g., wherein the multisink medium 400 is at least partially disposed between all or a portion of a transducer array 202 and all or a portion of another component of imaging device 100, 200, 250, such as a housing 231, a heatsink 268, a backing laminate, and/or an acoustic absorber 230). In some cases, transducer(s) 202 can be mounted on a substrate 260 and coupled to an acoustic absorber layer 230 (e.g., via one or more adhesive layers 262), optionally with a backing laminate (e.g., a metal reflector, such as an aluminum backing laminate or tungsten backing laminate, as shown in FIG. 3).

As described herein, multisink medium 400 can have properties uniquely advantageous for inclusion in imaging systems and devices 100, 200, 250. For instance, multisink medium 400 can be both thermally conductive and acoustically nonconductive (e.g., absorptive or dissipative of ultrasonic energy). In many cases, acoustic absorption layers 230, which often do not exhibit good thermal conduction, may be best used in imaging device 100, 200, 250 along with one or more heatsinks 268, which are often acoustically reflective. Multisink medium 400 can also be deformable or capable of flow (e.g., injectable or moldable), which can allow multisink medium 400 to be filled into cavities, gaps, or spaces 270 in an imaging device 100, 200, 250, allowing for more extensive acoustic isolation of components rearward of transducers 202 (e.g., disposed in an opposite direction than coating layer 222 and/or the target substance) and a larger contact area (e.g., cross-sectional area for thermal flux) between heat sensitive components of the imaging device 100, 200, 250 and exterior components (e.g., housing 231) and/or thermally conductive components that may be acoustically reflective (e.g., such as a heatsink 268). For instance, multisink medium 400 may allow more complete surrounding of acoustically reflective components of imaging device 100, 200, 250 than acoustic absorber layers 230, which may comprise a pad, a laminate, or a film and may not be injectable, moldable, or capable of flowing, e.g., into cavities, gaps, or spaces 270 around the acoustically reflective components. While multisink medium 400 is not explicitly shown in FIG. 3, FIG. 5, or FIG. 6, it is contemplated that multisink medium 400 one or more cavities, gaps, or spaces 270 of imaging devices 100, 200, 250 (e.g., such as those shown in FIG. 3, FIG. 5, and FIG. 6) can be partially or completely filled with multisink medium 400 and that some or all acoustic absorber layers 230 and/or heatsinks 268 described herein or depicted in the figures of this application may be replaced by multisink medium 400.

In some cases, imaging device 100, 200, 250 comprises multisink medium 400 and an acoustic absorber layer 230 (and, optionally, a backing laminate). While FIG. 3 depicts an acoustic absorber layer 230 and a backing laminate 232 (e.g., a metal backing, such as aluminum backing or a tungsten reflector), one or both of these components may be omitted or replaced (e.g., by multisink medium 400), for instance, where other components of imaging device 100, 200, 250 (e.g., multisink medium 400) substantially prevent transmission of ultrasound from transducers 202 in a direction away from coating layer 222. For example, imaging device 100, 200, 250 can comprise multisink medium 400 without an acoustic absorber layer 230 and, optionally, without a backing laminate, e.g., as shown in FIG. 4, FIG. 5, and FIG. 6. In some cases, use of multisink medium 400, e.g., in place of backing laminate 232, can simplify manufacture and/or reduce cost of components of imaging device 100, 200, 250.

FIG. 3 shows embodiments of an imaging device 250 comprising an acoustic absorption layer 230. As described herein, imaging devices 250 can comprise multisink medium 400, e.g., wherein the multisink medium is disposed within (e.g., partially or completely filling) cavities 270 and/or wherein multisink medium replaces one or more of backing laminate 232, adhesive layers 262, and/or acoustic absorber layer 230. As shown in FIG. 3, imaging device 250, which may be part of an ultrasound imaging system or device 100, 200 described herein, can comprise a coating layer 222, which may be a lens or may function as a lens. As shown in FIG. 3, coating layer 222 can be situated closer to a distal end of imaging device 250 as microelectromechanical (MEMs) transducer(s) 202, which may be coupled (e.g., directly) to ASIC 106. ASIC 106, which may comprise control circuitry can be coupled to a substrate 260 (e.g., an integrated circuit substrate, such as a printed circuit board (PCB)). ASIC 106 may include some or all electronic components of imaging device 250, which may include battery 238, memory 236, communication circuitry 232, processor 226, AFE 228, and/or port 234. In some cases, components of imaging device 250 (e.g., coating layer 222, transducer 202, ASIC 106, and/or substrate 260) may rest on or may be directly or indirectly coupled to one or more adhesive layers 262, absorber layer 230, and/or backing laminate 232, which may comprise a reflector, such as a tungsten reflector.

As shown in FIG. 4, multisink medium 400 can be disposed within one or more cavities, gaps, or spaces 270 of an imaging device 100, 200, 250. In some cases, multisink medium 400 can fill all or a portion of one or more cavities, gaps, or spaces 270 (e.g., cavities 270 shown in FIG. 5 or FIG. 6) of an imaging device 100, 200, 250. In some cases, multisink medium can be disposed between a housing 231 and a heatsink 268. In some cases, multisink medium can be in contact with a housing 231 of an imaging device 100, 200, 250. In some cases, multisink medium can be in contact with one or more heatsinks 268 of an imaging device 100, 200, 250. In some cases, multisink medium 400 can be disposed between one or more heatsinks 268 and a substrate 260 (e.g., which can comprise a printed circuit board) of an imaging device 100, 200, 250. In some cases, multisink medium 400 can be in contact with a substrate 260 of an imaging device 100, 200, 250. While not shown in contact in FIG. 4, multisink medium 400 can be disposed between an ASIC 106 (e.g., comprising control circuitry) and a heatsink 268 or housing 231 of an imaging device 100, 200, 250. In some cases, multisink medium 400 can be disposed between a transducer 202 (e.g., a transducer array, element, or pixel) and a heatsink 268 or housing 231 of an imaging device 100, 200, 250. It is understood that the cavities 270 of imaging device 250 shown in FIG. 6 can be partially or completely filled with multisink medium 400 and that one or more acoustic absorber layers 230 and/or one or more adhesive layers 262 and/or one or more backing laminates can be in contact with multisink medium 400, in some embodiments.

As shown in FIG. 3, FIG. 4, FIG. 5, and FIG. 6, a heatsink 268 and/or a housing may rest upon or be coupled directly to a substrate of imaging device 250, in accordance with various embodiments. In some cases, such configurations can provide one or more cavities, gaps, or spaces 270 in which multisink medium 400 can be disposed. In some cases, such configurations allow the heatsink 268 and/or the substrate 260 to mechanically support one or more components of imaging device 250 (e.g., rather than multisink medium 400).

In some cases, imaging device 100, 200, 250 can comprise a communication unit 232, e.g., for communicating data, including control signals, for example, with an external device such as a computing device (e.g., through a port 234 or wireless transceiver). Imaging device 100, 200, 250 can comprise a memory 236 (e.g., non-transitory memory), for example, for storing data and/or instructions for the operation of components of the imaging device (e.g., processors and/or transducers). Memory 236 can be embodied as a volatile or non-volatile memory or data storage (e.g., as described herein) capable of performing the functions described herein. In some cases, memory 236 may store various data and software that may be employed during operation of imaging device 100, 200, 250, such as operating systems, applications, programs, libraries, and/or drivers.

In some cases, imaging device 100, 200, 250 can include battery 238, e.g., for providing electrical power to one or more components of the imaging device, such as a transducer, a processor, and/or a memory. Battery 238 may include battery charging circuits, which may be wireless or wired charging circuits. Imaging device 100, 200, 250 may include a gauge that indicates battery charge consumed and may be used to configure the imaging device to optimize power management for improved battery life, in accordance with some embodiments.

Additionally or alternatively, imaging device 100, 200, 250 may be powered by an external power source (e.g., a plug for powering the imaging device from an electrical wall outlet), in accordance with some embodiments.

Imaging device 100, 200, 250 can comprise different suitable form factors, in various embodiments. In some embodiments a portion of the imaging device 100, 200, 250 that includes transducers 202 may extend outward from the rest of the imaging device 100, 200, 250. Imaging device 100, 200, 250 may be embodied as any suitable ultrasonic medical probe, such as a convex array probe, micro-convex array probe, linear array probe, endovaginal probe, endorectal probe, surgical probe, or intraoperative probe.

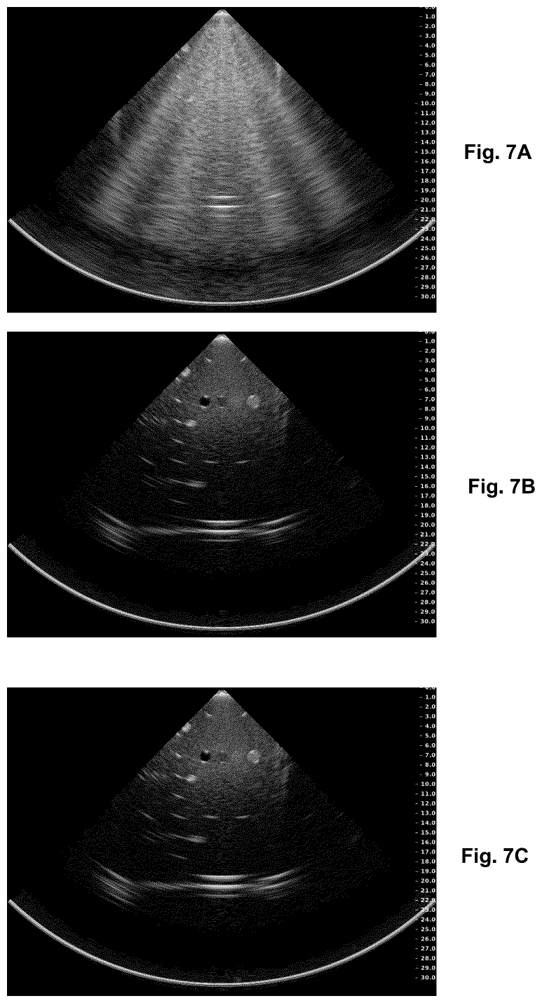

FIGS. 7A-7C show the effects of incorporation of multisink medium 400 (e.g., between a transducer and one or more additional components of an imaging system or device 100, 200, 250, such as a heatsink, metal backing, or housing) on scan quality and scan artifacts. FIG. 7A shows a scan of a test target substance (comprising pin targets, wire targets, and cysts), wherein a metal backing (in this case, an aluminum backing) is coupled to the integrated circuit substrate 260 of the imaging device 100, 200, 250 (e.g., via adhesives) utilized (e.g., to provide maximum heat transfer away from electronic components like ASICs and transducers). The scan shows significant reverberation artifacts; no test substance targets or features can be discerned due to washout. FIG. 7B shows a scan of the same test target substance using identical scan settings, wherein a multisink medium 400 is utilized between electronic components (including the integrated circuit substrate 260) and an aluminum heatsink. The scan shows clear features of all targets and features in the test substance with little or no reverberation artifacts. FIG. 7C shows a scan of the same test target substance using identical scan settings, wherein air backing is utilized between electronic components (including the integrated circuit substrate 260) and an aluminum heatsink. The scan shows clear features of all targets and features in the test substance with little or no reverberation artifacts.

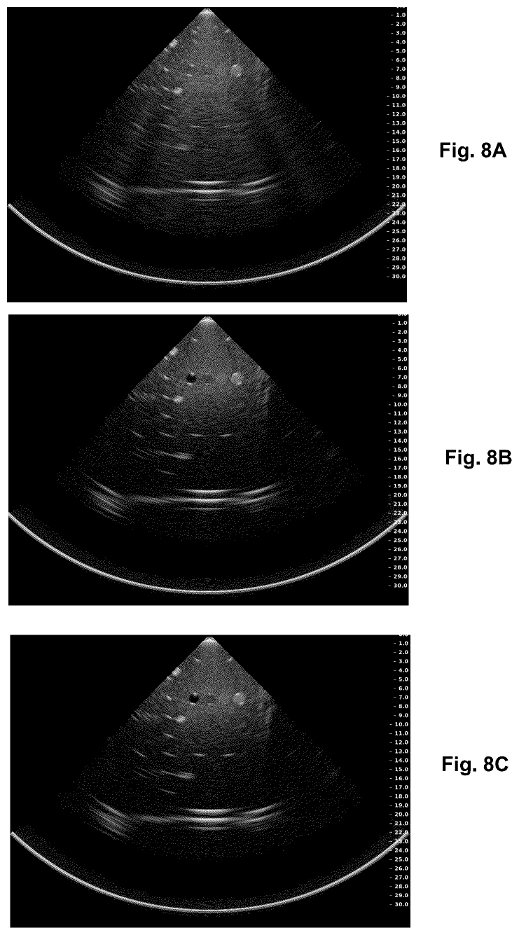

FIGS. 8A-8C show the effects of incorporation of varying thicknesses of multisink medium 400 (e.g., between a transducer and one or more additional components of an imaging system or device 100, 200, 250, such as a heatsink, metal backing, or housing) on scan quality on scan artifacts. FIG. 8A shows a scan of a test target substance (comprising pin targets, wire targets, and cysts) at a center frequency of 5.1 MHz using an imaging device 100 comprising multisink medium 400 having a thickness of 0.5 mm disposed between an integrated circuit substrate 260 and an aluminum heatsink of the imaging device. The scan shows clearly discernible test substance targets and features with only minor reverberation artifact striations in the image. These results show a significant improvement over scan results without multisink medium 400 (e.g., as shown in FIG. 7A). FIG. 8B shows a scan of the same test substance using identical scan settings and instrument conditions except for the thickness of the multisink medium 400, which is 1.0 mm. The scan shows a significant improvement over scan results shown in FIG. 8A, the increased thickness of multisink medium 400 resulting in an image with clear features of all targets and features in the test substance with little or no reverberation artifacts. FIG. 8C shows a scan of the same test substance using identical scan settings and instrument conditions except the multisink medium 400 is replaced by air backing (e.g., an air-filled gap between a transducer and a heatsink of the imaging device 100 instead of a multisink medium 400-filled gap). The scan shows similar results to those shown in FIG. 8B, with clear features of all targets and features in the test substance apparent in the image, with little or no reverberation artifacts. In all, these results show that a multisink medium 400 thickness of 0.5 mm or less is sufficient to improve ultrasound scan quality (e.g., reduce reverberation artifacts), with quality improving (e.g., as a result of reduced internal acoustic reflection and reverberation artifacts) to ideal conditions at a multisink medium 400 thickness of as little as 1.0 mm. These results suggest that a multisink medium 400 thickness of 1.5 mm (e.g., +/−0.25 mm) is sufficient to attenuate or eliminate all reverberation artifacts from any scan condition at midrange or high center frequencies.

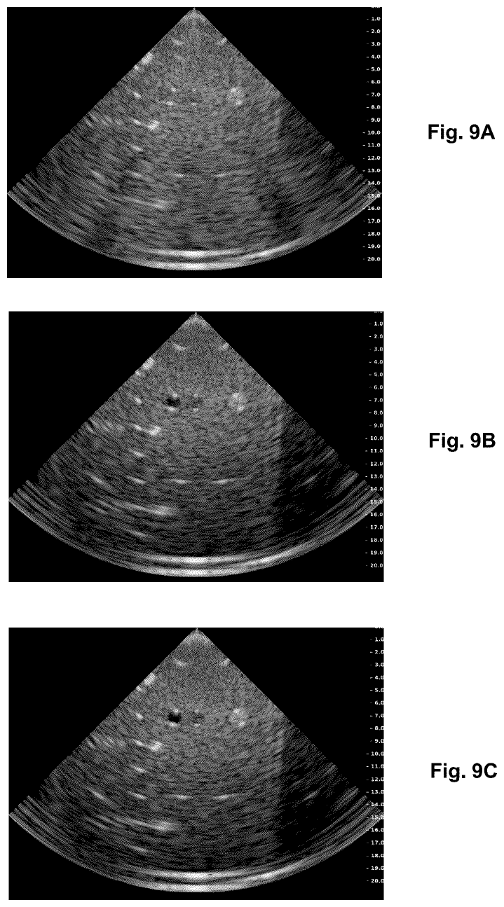

FIGS. 9A-9C show the effects of incorporation of varying thicknesses of multisink medium 400 (e.g., between a transducer and one or more additional components of an imaging system or device 100, 200, 250, such as a heatsink, metal backing, or housing) on scan quality on scan artifacts, using a scan having a lower center frequency. FIG. 9A shows a scan of a test target substance (comprising pin targets, wire targets, and cysts) at a center frequency of 1.8 MHZ using an imaging device 100 comprising multisink medium 400 having a thickness of 0.5 mm disposed between an integrated circuit substrate 260 and an aluminum heatsink of the imaging device. The scan shows clearly discernible test substance targets and features with only minor reverberation artifact striations in the image. These results show a significant improvement over scan results without multisink medium 400 (e.g., as shown in FIG. 7A). FIG. 9B shows a scan of the same test substance using identical scan settings and instrument conditions except for the thickness of the multisink medium 400, which is 1.0 mm. The scan shows a significant improvement over scan results shown in FIG. 9A, the increased thickness of multisink medium 400 resulting in an image with clear features of all targets and features in the test substance with little or no reverberation artifacts. FIG. 9C shows a scan of the same test substance using identical scan settings and instrument conditions except the multisink medium 400 is replaced by air backing (e.g., an air-filled gap between a transducer and a heatsink of the imaging device 100 instead of a multisink medium 400-filled gap). The scan shows similar results to those shown in FIG. 9B, with clear features of all targets and features in the test substance apparent in the image, with little or no reverberation artifacts. In all, these results show that a multisink medium 400 thickness of 0.5 mm or less is sufficient to improve ultrasound scan quality (e.g., reduce reverberation artifacts), with quality improving (e.g., as a result of reduced internal acoustic reflection and reverberation artifacts) to ideal conditions at a multisink medium 400 thickness of as little as 1.0 mm. These results suggest that a multisink medium 400 thickness of 1.5 mm (e.g., +/−0.25 mm) is sufficient to attenuate or eliminate all reverberation artifacts from any scan condition at low center frequencies.

### Transducers

In some cases, systems, devices, or methods described herein can comprise one or more piezoelectric micromachine ultrasound transducers (pMUTs). In some cases, system, devices, or methods described herein can comprise one or more capacitive micromachine ultrasonic transducers (cMUTs). Piezoelectric micromachine ultrasound transducers (pMUTs) can be formed on a substrate 260, such as a semiconductor wafer (e.g., a printed circuit board, PCB). pMUT elements constructed on semiconductor substrates 260 can offer a smaller size profile than bulky conventional transducers having bulkier piezoelectrical material. In some cases, pMUTs can also be less expensive to manufacture and/or may allow less complicated and higher performance interconnection between the transducers and additional electronics of the ultrasound device or system.

Micromachine ultrasound transducers (MUTs), which can include pMUTs and/or cMUTs can include a diaphragm (e.g., a thin membrane attached, for example at the membrane edges, to one or more portions of the interior of an imaging device (e.g., ultrasound probe)). In contrast, traditional bulk piezoelectric (PZT) elements typically consist of a single solid piece of material. Such traditional PZT ultrasound systems and devices can be expensive to fabricate, for example, because great precision is required to cut and mount PZT or ceramic material comprising the PZT ultrasound systems and devices with the proper spacing. Additionally, traditional PZT ultrasound systems and devices can have significantly higher transducer impedance compared to the impedance of the transmit/receive electronics of the PZT systems and devices, which can adversely affect performance.

In some cases, one or more transducer elements 104 can be configured to transmit and/or receive signals at a specific frequency or bandwidth (e.g., wherein the bandwidth is associated with a center frequency). In some cases, one or more transducer elements can be further configured to transmit and/or receive signals at additional center frequencies and bandwidths. Such multi-frequency transducer elements 104 can be referred to as multi-modal elements 104, and can, in some embodiments, be used to expand a bandwidth of an imaging system or device 100. A transducer element or pixel 104 can be configured to emit (e.g., transmit) and/or receive an ultrasonic energy (e.g., an ultrasonic waveform, pattern, or pressure wave) at a suitable center frequency, e.g., from 0.1 megahertz (MHz) to 100 MHz. In some cases, a transducer or pixel 104 can be configured to transmit or receive ultrasonic energy at a center frequency of 0.1 MHz to 1 MHz, 0.1 MHz to 1.8 MHz, 0.1 MHz to 3.5 MHz, 0.1 MHz to 5.1 MHz, 0.1 MHz to 10 MHZ, 0.1 MHz to 25 MHz, 0.1 MHz to 50 MHz, 0.1 MHz to 100 MHz, 1 MHz to 1.8 MHz, 1 MHz to 3.5 MHz, 1 MHz to 5.1 MHz, 1 MHz to 10 MHz, 1 MHz to 25 MHz, 1 MHz to 50 MHz, 1 MHZ to 100 MHz, 1.8 MHz to 3.5 MHz, 1.8 MHz to 5.1 MHz, 1.8 MHz to 10 MHz, 1.8 MHz to 25 MHz, 1.8 MHz to 50 MHz, 1.8 MHz to 100 MHZ, 3.5 MHz to 5.1 MHz, 3.5 MHz to 10 MHZ, 3.5 MHz to 25 MHz, 3.5 MHz to 50 MHz, 3.5 MHz to 100 MHz, 5.1 MHz to 10 MHz, 5.1 MHZ to 25 MHZ, 5.1 MHz to 50 MHz, 5.1 MHz to 100 MHz, 10 MHz to 25 MHz, 10 MHz to 50 MHz, 10 MHz to 100 MHz, 25 MHz to 50 MHz, 25 MHz to 100 MHz, or 50 MHz to 100 MHZ.

In some cases, a transducer or pixel 104 can be configured to transmit or receive ultrasonic energy at a center frequency of 0.1 MHZ, 1 MHZ, 1.8 MHz, 3.5 MHz, 5.1 MHZ, 10 MHZ, 25 MHz, 50 MHz, or 100 MHz. In some cases, a transducer or pixel 104 can be configured to transmit or receive ultrasonic energy at a center frequency of at least 0.1 MHZ, 1 MHZ, 1.8 MHZ, 3.5 MHz, 5.1 MHz, 10 MHz, 25 MHz, 50 MHz, or 100 MHz. In some cases, a transducer or pixel 104 can be configured to transmit or receive ultrasonic energy at a center frequency of at most 0.1 MHz, 1 MHz, 1.8 MHz, 3.5 MHz, 5.1 MHz, 10 MHZ, 25 MHz, 50 MHz, or 100 MHz.

### Multisink Medium 400

An imaging device or system (e.g., an ultrasound imaging device or system) described herein can comprise a multisink medium 400. A multisink medium 400 can be acoustically nonconductive (e.g., nonconductive of ultrasonic energy). A multisink medium 400 can be acoustically nonreflective (e.g., nonreflective of ultrasonic energy). For example, multisink medium 400 can partially or completely inhibit conduction and/or reflection of acoustic energy (e.g., ultrasonic energy), in some embodiments. In some cases, a multisink medium 400 can partially or completely absorb or otherwise dissipate acoustic energy (e.g., ultrasonic energy). A multisink medium 400 can be thermally conductive. A multisink medium 400 can comprise a substance that is both thermally conductive and acoustically nonconductive.

In many cases, a multisink medium 400 can absorb some or all energy of an incident ultrasound waveform or pattern (e.g., acoustically nonconductive). For example, a multisink medium 400 can comprise a substance capable of reducing the energy of an incident ultrasound waveform or pattern. In many cases, a multisink medium 400 can comprise a substance capable of absorbing all or a portion of incident acoustic energy produced by an ultrasound transducer of an imaging device or system described herein. In some cases, multisink medium 400 is disposed between an ultrasound transducer and an acoustically reflective material, such as a housing 231, a heatsink 268, and/or a substrate 260 of an imaging device or system, for example, to decrease the transmission of acoustic energy reflected toward a transducer of the imaging device or system from a substance other than a target substance or portion thereof (e.g., acoustic energy reflected from an acoustically reflective material comprising a portion of the imaging device or system). In some cases, substances comprising metals can be acoustically reflective. In some cases, a multisink medium 400 can be free of metals. In some cases, a multisink medium 400 can be free of metal oxides. For example, a multisink medium 400 may not comprise metal particles (e.g., aluminum particles), in some embodiments.

A multisink medium 400 can comprise a paste or a putty. In some cases, a multisink medium 400 can comprise a filler. In some cases, a multisink medium 400 can comprise a tape, sheet, or film. In some cases, a multisink medium 400 can comprise a pad.

In some cases, a multisink medium 400 can comprise an elastomer. For example, a multisink medium 400 can comprise a silicone elastomer or a silicone-based elastomer. In some cases, a silicone elastomer or silicone-based elastomer can combine desirable deformability properties (e.g., an ability to flow and/or to be injected or extruded at room temperature) with high properties of high thermal conductivity (e.g., which may arise from the thermal conductivity of silicone). A multisink medium 400 can comprise a material capable of inhibiting, at least in part, propagation of ultrasound energy. In some cases, a multisink medium 400 can comprise a material capable of absorbing, at least in part, ultrasound energy. For example, a multisink medium 400 can comprise a ceramic material. In some cases, a multisink medium 400 can comprise a ceramic-filled silicone elastomer (or ceramic-filled silicone-based elastomer). In some cases, a multisink medium 400 can comprise particles dispersed within the bulk of the multisink medium 400. For example, a multisink medium 400 can comprise a silicone elastomer comprising ceramic particles. In some cases, a silicone-based elastomer comprising ceramic particles can be a paste or putty.

In some cases, a multisink medium 400 is capable of deforming or flowing (e.g., at room temperature). A multisink medium 400 that is deformable or capable of flowing at room temperature (e.g., injectable at room temperature) can be useful in filling irregularly shaped geometries within an imaging system or device 100, 200, 250 (e.g., within a cavity within a probe of an imaging device or system, such as a cavity completely or partially surrounding a heatsink of the imaging device or system). In some cases, a multisink medium 400 can be injected into a cavity, gap, or space 270 of an imaging system or device 100, 200, 250 or portion thereof (e.g., wherein the multisink medium 400 contacts a heatsink and/or a printed circuit board). In some embodiments, a multisink medium 400 can be added to an imaging device or system or portion thereof (e.g., in contact with a heatsink and/or printed circuit board of an imaging device or system probe). For example, a multisink medium 400 can be injectable or moldable (e.g., at room temperature). In some cases, a multisink medium 400 can have a flow rate of 25 grams per minute (g/min) to 45 g/min. In some cases, a multisink medium 400 can have a flow rate of 25 g/min to 30 g/min, 25 g/min to 35 g/min, 25 g/min to 40 g/min, 25 g/min to 45 g/min, 30 g/min to 35 g/min, 30 g/min to 40 g/min, 30 g/min to 45 g/min, 35 g/min to 40 g/min, 35 g/min to 45 g/min, or 40 g/min to 45 g/min. In some cases, a multisink medium 400 can have a flow rate of 25 g/min, 30 g/min, 35 g/min, 40 g/min, or 45 g/min. In some cases, a multisink medium 400 can have a flow rate of at least 25 g/min, at least 30 g/min, at least 35 g/min, at least 40 g/min, at least 45 g/min. In some cases, a multisink medium 400 can have a flow rate of at most 30 g/min, at most 35 g/min, at most 40 g/min, or at most 45 g/min. In some cases, a multisink medium 400 has a non-Newtonian viscosity (e.g., having shear thinning properties). In some cases, a multisink medium 400 has time-dependent viscosity (e.g., having thixotropic properties). In some cases, a multisink medium 400 that is or that comprises an elastomer can be deformable or capable of flowing. For example, a multisink medium 400 that is an elastomer can be injectable or moldable, in some embodiments.

In some cases, a multisink medium 400 that is deformable or capable of flowing (e.g., injectable or moldable) can allow heat transfer to be improved in portions of an imaging system or imaging device in which it would not otherwise be easy or, potentially, possible to improve heat transfer. For example, heat transfer in imaging systems and imaging devices or portions thereof (e.g., an ultrasound transducer probe), especially those that benefit from reduced or miniaturized overall dimensions (e.g., in a handheld ultrasound transducer probe head), is often facilitated by traditional heatsinks (e.g., metal heatsinks, such as aluminum heatsink blocks), which may be supported by, attached to, in contact with or in proximity but not in contact with other, potentially heat-sensitive components of the imaging system or device, may need to be fabricated separately, making (e.g., air-filled) cavities, gaps, or spaces in the imaging system or device or portion thereof likely or necessary. Such cavities, gaps, or spaces (e.g., when filled with a thermal insulator, such as a gas, e.g., air) can reduce the heat transfer within the imaging system or imaging device. Traditional heatsinks (e.g., metal heatsinks, such as aluminum heatsink blocks) may not easily be fabricated to fill such cavities, gaps, or spaces. Thus, inclusion of a multisink medium 400 capable of being molded, pressed, injected, deformed or otherwise allowed to conformed into all or a portion of a cavity, gap, or space of an imaging system or device can improve the heat transfer in regions of imaging systems and devices that traditional heat transfer components may not be able to occupy. In some cases, a multisink (e.g., disposed within an imaging system or device or within a cavity, gap, or space 270 thereof) can have a thickness of 0.5 (millimeters) mm to 2.0 mm. In some cases, a multisink (e.g., disposed within an imaging system or device or within a cavity, gap, or space 270 thereof) can have a thickness of 0.5 millimeters (mm) to 0.8 mm, 0.5 mm to 1.0 mm, 0.5 mm to 1.3 mm, 0.5 mm to 1.5 mm, 0.5 mm to 1.7 mm, 0.5 mm to 2 mm, 0.8 mm to 1.0 mm, 0.8 mm to 1.3 mm, 0.8 mm to 1.5 mm, 0.8 mm to 1.7 mm, 0.8 mm to 2 mm, 1.0 mm to 1.3 mm, 1.0 mm to 1.5 mm, 1.0 mm to 1.7 mm, 1.0 mm to 2.0 mm, 1.3 mm to 1.5 mm, 1.3 mm to 1.7 mm, 1.3 mm to 2 mm, 1.5 mm to 1.7 mm, 1.5 mm to 2.0 mm, or 1.7 mm to 2.0 mm. In some cases, a multisink (e.g., disposed within an imaging system or device or within a cavity, gap, or space 270 thereof) can have a thickness of 0.5 mm, 0.8 mm, 1.0 mm, 1.3 mm, 1.5 mm, 1.7 mm, or 2.0 mm. In some cases, a multisink (e.g., disposed within an imaging system or device or within a cavity, gap, or space 270 thereof) can have a thickness of at least 0.5 mm, at least 0.8 mm, at least 1.0 mm, at least 1.3 mm, at least 1.5 mm, at least 1.7 mm, or at least 2.0 mm. In some cases, a multisink medium (e.g., disposed within an imaging system or device or within a cavity, gap, or space 270 thereof) can have a thickness of at most 0.8 mm, at most 1.0 mm, at most 1.3 mm, at most 1.5 mm, at most 1.7 mm, or at most 2.0 mm. In some cases, reverberation artifacts can be completely eliminated from ultrasound scans when multisink medium of the imaging system or device 100, 200, 250 has a thickness of at least 1.0 mm or at least 1.5 mm, in some cases with a tolerance of +/−0.25 mm. In some cases, a method of manufacturing an imaging system or device 100, 200, 250 can comprise injecting or molding multisink medium 400 to partially or completely fill one or more cavities, gaps, or spaces 270 of the imaging system or device. In some cases, an imaging system or device 100, 200, 250 can be provided with a fully captured edge (e.g., wherein the edges of one or more cavities, gaps, or spaces 270 of the imaging system or device are sealed together when coupled, for instance to prevent seepage of multisink medium 400 when injected). In some cases, an imaging system or device 100, 200, 250 can be provided with one or more injection ports (e.g., through one or more heatsinks of the imaging system or device) to facilitate injection of multisink medium 400 into one or more cavities, gaps, or spaces 270 of the imaging system or device. In some cases, an injection port is provided through the side of a housing or heatsink (e.g., from an exterior side of the heatsink to an interior side of the heatsink open to a cavity, gap, or space of the imaging system or device, for example, to avoid interfering with the bonding between a substrate 260 and the heatsink). In some cases, multisink medium 400 can be added to an imaging device or system before a heatsink and substrate or housing and substrate are coupled/bonded, e.g., to entrap the multisink medium 400 within cavities, gaps, or spaces when the heatsink is bonded to the substrate.

In some cases, a multisink medium 400 can be thermally conductive. For example, a multisink medium 400 can have a thermal conductivity of 2 Watts per meter-Kelvin (W/mK) to 7 W/mK. In some cases, a multisink medium 400 can have a thermal conductivity of 2 W/mK to 2.3 W/mK, 2 W/mK to 2.5 W/mK, 2 W/mK to 3 W/mK, 2 W/mK to 3.7 W/mK, 2 W/mK to 4 W/mK, 2 W/mK to 5 W/mK, 2 W/mK to 5.5 W/mK, 2 W/mK to 6 W/mK, 2 W/mK to 6.4 W/mK, 2 W/mK to 7 W/mK, 2.3 W/mK to 2.5 W/mK, 2.3 W/mK to 3 W/mK, 2.3 W/mK to 3.7 W/mK, 2.3 W/mK to 4 W/mK, 2.3 W/mK to 5 W/mK, 2.3 W/mK to 5.5 W/mK, 2.3 W/mK to 6 W/mK, 2.3 W/mK to 6.4 W/mK, 2.3 W/mK to 7 W/mK, 2.5 W/mK to 3 W/mK, 2.5 W/mK to 3.7 W/mK, 2.5 W/mK to 4 W/mK, 2.5 W/mK to 5 W/mK, 2.5 W/mK to 5.5 W/mK, 2.5 W/mK to 6 W/mK, 2.5 W/mK to 6.4 W/mK, 2.5 W/mK to 7 W/mK, 3 W/mK to 3.7 W/mK, 3 W/mK to 4 W/mK, 3 W/mK to 5 W/mK, 3 W/mK to 5.5 W/mK, 3 W/mK to 6 W/mK, 3 W/mK to 6.4 W/mK, 3 W/mK to 7 W/mK, 3.7 W/mK to 4 W/mK, 3.7 W/mK to 5 W/mK, 3.7 W/mK to 5.5 W/mK, 3.7 W/mK to 6 W/mK, 3.7 W/mK to 6.4 W/mK, 3.7 W/mK to 7 W/mK, 4 W/mK to 5 W/mK, 4 W/mK to 5.5 W/mK, 4 W/mK to 6 W/mK, 4 W/mK to 6.4 W/mK, 4 W/mK to 7 W/mK, 5 W/mK to 5.5 W/mK, 5 W/mK to 6 W/mK, 5 W/mK to 6.4 W/mK, 5 W/mK to 7 W/mK, 5.5 W/mK to 6 W/mK, 5.5 W/mK to 6.4 W/mK, 5.5 W/mK to 7 W/mK, 6 W/mK to 6.4 W/mK, 6 W/mK to 7 W/mK, or 6.4 W/mK to 7 W/mK. In some cases, a multisink medium 400 can have a thermal conductivity of 2 W/mK, 2.3 W/mK, 2.5 W/mK, 3 W/mK, 3.7 W/mK, 4 W/mK, 5 W/mK, 5.5 W/mK, 6 W/mK, 6.4 W/mK, or 7 W/mK. In some cases, a multisink medium 400 can have a thermal conductivity of at least 2 W/mK, at least 2.3 W/mK, at least 2.5 W/mK, at least 3 W/mK, at least 3.7 W/mK, at least 4 W/mK, at least 5 W/mK, at least 5.5 W/mK, at least 6 W/mK, at least 6.4 W/mK, or at least 7.0 W/mK. In some cases, a multisink medium 400 can have a thermal conductivity of at most 2.3 W/mK, at most 2.5 W/mK, at most 3 W/mK, at most 3.7 W/mK, at most 4 W/mK, at most 5 W/mK, at most 5.5 W/mK, at most 6 W/mK, at most 6.4 W/mK, or at most 7 W/mK. In some cases, selection of a matrix material can determine whether a multisink medium 400 is thermally conductive. In some cases, a multisink medium 400 can be thermally conductive when the multisink medium 400 comprises a silicone elastomer or a silicone-based elastomer. A multisink medium 400 that is thermally conductive can improve heat transfer in an imaging device or imaging system. For example, a multisink medium 400 (e.g., in contact with a component of an imaging device or imaging system, such as an integrated circuit (e.g., ASIC), processor, and/or a transducer element) can aid in transferring heat away from one or more components that may produce heat (e.g., during operation) and/or may be sensitive to excessive heat (e.g., which may overheat, experience reduced performance, and/or cease to function in the presence of excessive heat). In some cases, a multisink medium 400 can facilitate heat transfer by contacting another thermally conductive component of an imaging system or imaging device, such as a heatsink and/or a housing or casing of the imaging system or imaging device. In some cases, a heatsink that is thermally conductive can be located, positioned, injected, or deposited in a cavity, gap, or space of an imaging system or imaging device or a portion thereof, e.g., to improve heat transfer (e.g., away from one or more heat sensitive components of the imaging system or imaging device). In some cases, a cavity, gap, or space of an imaging system or device may result from a method used to fabricate the imaging system or device or may be included in the design of the system or device to help electrically or mechanically isolate a first component of the system or device from a second component of the system or device. In some cases, a multisink located, positioned, injected, or deposited in a cavity, gap, or space of an imaging system or imaging device or portion thereof can replace a component or material (e.g., a gas, such as air) otherwise located in the cavity, gap, or space, e.g., wherein the replaced component or material has a lower thermal conductivity than the multisink medium 400. For example, filling a cavity, gap, or space of an imaging system or device with a multisink medium 400 can replace a less thermally conductive component or material (e.g., air) and can increase heat transfer within the system or device, which may reduce the risk of overheating or heat-related reductions in performance of the imaging system or imaging device. In some cases, a multisink medium 400 is both thermally conductive (e.g., as described herein) and acoustically non-conductive (e.g., acoustically insulative), as many thermally conductive materials (e.g., aluminum heatsinks) can cause artifacts during ultrasound scanning, for example, as a result of acoustic reverberation arising from the presence of the thermally conductive material's tendency to reflect ultrasound waves.

### Backing Layers

An imaging system or device 100 (e.g., an ultrasound imaging system or device 100) described herein can comprise a backing layer 232 (e.g., a backing laminate). In some cases, a backing layer 232 (e.g., backing laminate), for example, comprising an absorber layer 230 and/or a multisink medium 400, can be added to an imaging system or device 100 (e.g., positioned inside of a probe of an imaging system or device 100) to decrease reverberation artifacts, e.g., which may result from reverberation of ultrasound energy within the imaging system or device 100. In some cases, an imaging system or device 100 can comprise a multisink medium 400 and a backing layer 232. A backing layer 232 (e.g., a backing laminate) can comprise a metal. For example, a backing laminate 232 can comprise a tungsten reflector. In some cases, an absorber layer 230, which can in some cases comprise a portion of a backing layer 232, can comprise a metal foam (e.g., copper foam). In some cases, a backing layer (e.g., a backing laminate) can comprise an aluminum backing. In some cases, a backing layer can comprise one or more adhesive layers 262 (e.g., for coupling the components of the backing layer together and/or to one or more components of an imaging system or device 100, 200, 250, such as a substrate 260 and/or a heatsink 268).

### Computing System

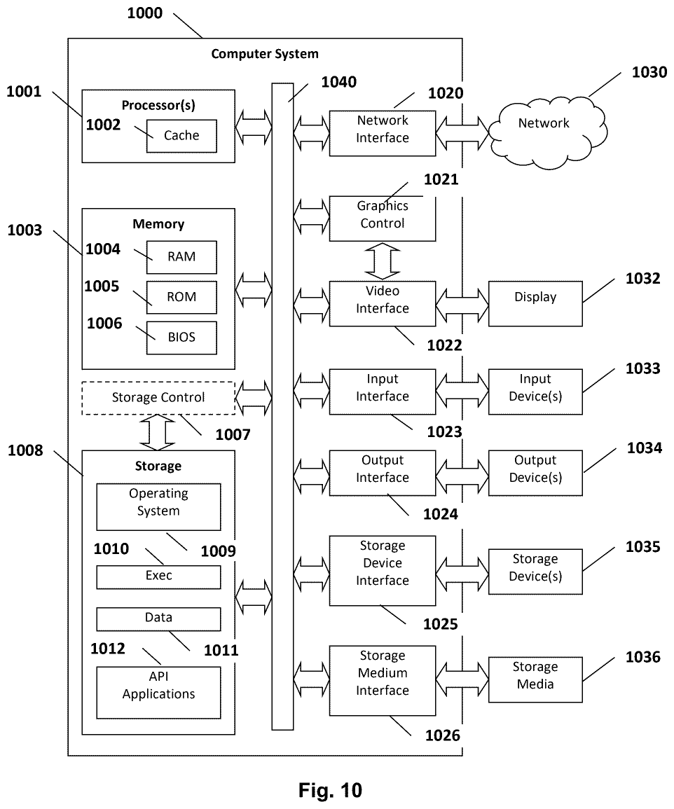

Referring to FIG. 10, a block diagram is shown depicting an exemplary machine that includes a computer system 1000 (e.g., a processing or computing system) within which a set of instructions can execute for causing a device to perform or execute any one or more of the aspects and/or methodologies for static code scheduling of the present disclosure. The components in FIG. 10 are examples only and do not limit the scope of use or functionality of any hardware, software, embedded logic component, or a combination of two or more such components implementing particular embodiments.

Computer system 1000 may include one or more processors 1001, a memory 1003, and a storage 1008 that communicate with each other, and with other components, via a bus 1040. The bus 1040 may also link a display 1032, one or more input devices 1033 (which may, for example, include a keypad, a keyboard, a mouse, a stylus, etc.), one or more output devices 1034, one or more storage devices 1035, and various tangible storage media 1036. All of these elements may interface directly or via one or more interfaces or adaptors to the bus 1040. For instance, the various tangible storage media 1036 can interface with the bus 1040 via storage medium interface 1026. Computer system 1000 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1000 includes one or more processor(s) 1001 (e.g., central processing units (CPUs), general purpose graphics processing units (GPGPUs), or quantum processing units (QPUs)) that carry out functions. Processor(s) 1001 optionally contains a cache memory unit 1002 for temporary local storage of instructions, data, or computer addresses. Processor(s) 1001 are configured to assist in execution of computer readable instructions. Computer system 1000 may provide functionality for the components depicted in FIG. 10 as a result of the processor(s) 1001 executing non-transitory, processor-executable instructions embodied in one or more tangible computer-readable storage media, such as memory 1003, storage 1008, storage devices 1035, and/or storage medium 1036. The computer-readable media may store software that implements particular embodiments, and processor(s) 1001 may execute the software. Memory 1003 may read the software from one or more other computer-readable media (such as mass storage device(s) 1035, 1036) or from one or more other sources through a suitable interface, such as network interface 1020. The software may cause processor(s) 1001 to carry out one or more processes or one or more steps of one or more processes described or illustrated herein. Carrying out such processes or steps may include defining data structures stored in memory 1003 and modifying the data structures as directed by the software.

The memory 1003 may include various components (e.g., machine readable media) including, but not limited to, a random access memory component (e.g., RAM 1004) (e.g., static RAM (SRAM), dynamic RAM (DRAM), ferroelectric random access memory (FRAM), phase-change random access memory (PRAM), etc.), a read-only memory component (e.g., ROM 1005), and any combinations thereof. ROM 1005 may act to communicate data and instructions unidirectionally to processor(s) 1001, and RAM 1004 may act to communicate data and instructions bidirectionally with processor(s) 1001. ROM 1005 and RAM 1004 may include any suitable tangible computer-readable media described below. In one example, a basic input/output system 1006 (BIOS), including basic routines that help to transfer information between elements within computer system 1000, such as during start-up, may be stored in the memory 1003.

Fixed storage 1008 is connected bidirectionally to processor(s) 1001, optionally through storage control unit 1007. Fixed storage 1008 provides additional data storage capacity and may also include any suitable tangible computer-readable media described herein. Storage 1008 may be used to store operating system 1009, executable(s) 1010, data 1011, applications 1012 (application programs), and the like. Storage 1008 can also include an optical disk drive, a solid-state memory device (e.g., flash-based systems), or a combination of any of the above. Information in storage 1008 may, in appropriate cases, be incorporated as virtual memory in memory 1003.

In one example, storage device(s) 1035 may be removably interfaced with computer system 1000 (e.g., via an external port connector (not shown)) via a storage device interface 1025. Particularly, storage device(s) 1035 and an associated machine-readable medium may provide non-volatile and/or volatile storage of machine-readable instructions, data structures, program modules, and/or other data for the computer system 1000. In one example, software may reside, completely or partially, within a machine-readable medium on storage device(s) 1035. In another example, software may reside, completely or partially, within processor(s) 1001.

Bus 1040 connects a wide variety of subsystems. Herein, reference to a bus may encompass one or more digital signal lines serving a common function, where appropriate. Bus 1040 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1000 may also include an input device 1033. In one example, a user of computer system 1000 may enter commands and/or other information into computer system 1000 via input device(s) 1033. Examples of an input device(s) 1033 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1033 may be interfaced to bus 1040 via any of a variety of input interfaces 1023 (e.g., input interface 1023) including, but not limited to, serial, parallel, game port, USB, FIREWIRE, THUNDERBOLT, or any combination of the above.

In particular embodiments, when computer system 1000 is connected to network 1030, computer system 1000 may communicate with other devices, specifically mobile devices and enterprise systems, distributed computing systems, cloud storage systems, cloud computing systems, and the like, connected to network 1030. Communications to and from computer system 1000 may be sent through network interface 1020. For example, network interface 1020 may receive incoming communications (such as requests or responses from other devices) in the form of one or more packets (such as Internet Protocol (IP) packets) from network 1030, and computer system 1000 may store the incoming communications in memory 1003 for processing. Computer system 1000 may similarly store outgoing communications (such as requests or responses to other devices) in the form of one or more packets in memory 1003 and communicated to network 1030 from network interface 1020. Processor(s) 1001 may access these communication packets stored in memory 1003 for processing.

Examples of the network interface 1020 include, but are not limited to, a network interface card, a modem, and any combination thereof. Examples of a network 1030 or network segment 1030 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1030, may employ a wired and/or a wireless mode of communication. In general, any network topology may be used.

Information and data can be displayed through a display 1032. Examples of a display 1032 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1032 can interface to the processor(s) 1001, memory 1003, and fixed storage 1008, as well as other devices, such as input device(s) 1033, via the bus 1040. The display 1032 is linked to the bus 1040 via a video interface 1022, and transport of data between the display 1032 and the bus 1040 can be controlled via the graphics control 1021. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1032, computer system 1000 may include one or more other peripheral output devices 1034 including, but not limited to, an audio speaker, a printer, a storage device, and any combinations thereof. Such peripheral output devices may be connected to the bus 1040 via an output interface 1024. Examples of an output interface 1024 include, but are not limited to, a serial port, a parallel connection, a USB port, a FIREWIRE port, a THUNDERBOLT port, and any combinations thereof.

In addition or as an alternative, computer system 1000 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, handheld computers, Internet appliances, mobile smartphones, and tablet computers. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

### Applications

In some cases, an imaging system or device 100 described herein can be used in (e.g., non-invasive) medical imaging, lithotripsy, localized tissue heating for therapeutic interventions, highly intensive focused ultrasound (HIFU) surgery, and/or non-medical uses flow measurements in pipes (or speaker and microphone arrays). In some cases, an imaging system or device described herein can be used to determine direction and/or velocity of fluid flow (e.g., blood flow) in arteries and/or veins, for example using Doppler mode imaging. In some cases, an imaging system or device described herein can be used to measure tissue stiffness.

In some cases, an imaging system or device 100 described herein can be configured to perform one-dimensional imaging (e.g., A-Scan imaging). In some cases, an imaging system or device 100 described herein can be configured to perform two-dimensional imaging (e.g., B-Scan imaging). In some cases, an imaging system or device 100 described herein can be configured to perform three-dimensional imaging (e.g., C-Scan imaging). In some cases, an imaging system or device 100 described herein can be configured to perform Doppler imaging. In some cases, an imaging system or device 100 described herein may be switched to a different mode (e.g., between modes), including linear mode or sector mode. In some cases, an imaging system or device 100 can be electronically configured under program control (e.g., by a user).

In many cases, an imaging system or device 100 (e.g., a probe of an imaging system or device 100) can be portable. For instance, an imaging system or device 100 can comprise (e.g., house within a housing) a handheld casing, which can house one or more transducer elements, pixels, or arrays, ASICs, control circuitry, and/or a computing device. In some case, an imaging system or device 100 can comprise a battery.

### Certain Definitions

Unless otherwise defined, all technical terms used herein have the same meaning as commonly understood by one of ordinary skill in the art to which the present subject matter belongs.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1. (canceled)

2. An imaging device comprising:
an integrated circuit substrate;
one or more microelectromechanical (MEMs) ultrasound transducers coupled to the integrated circuit substrate; and
an injectable or moldable multisink medium that is thermally conductive and acoustically non-conductive, the multisink medium being in contact with the integrated circuit substrate and filled into cavities, gaps, or spaces between the integrated circuit substrate and a heatsink, wherein the multisink medium is in contact with the heatsink and reduces acoustic reflections reflecting off the heatsink.

3. The device of claim 2, wherein the multisink medium is in contact with the heatsink.

4. The device of claim 2, wherein the multisink medium is disposed at least partially between the one or more MEMs transducers and the heatsink.

5. The device of claim 2, wherein the device further comprises a housing coupled to the integrated circuit substrate.

6. The device of claim 5, wherein the multisink medium is in contact with the housing.

7. The device of claim 5, wherein the multisink medium is disposed at least partially between the one or more MEMs transducers and the housing.

8. The device of claim 2, wherein the multisink medium has a flow rate of at least 29 g/min.

9. The device of claim 2, wherein the multisink medium has a thermal conductivity of at least 1.5 Watts per meter-Kelvin (W/mK).

10. The device of claim 2, wherein the multisink medium has a thickness of at least 0.5 mm.

11. The device of claim 2, wherein the multisink medium is injected into the gaps and flowed between the integrated circuit substrate and the one or more MEMs ultrasound transducers.

12. The device of claim 2, wherein at least one of the cavities, gaps, or spaces that the multisink medium fills extends at least an entire length of the one or more MEMs ultrasound transducers.

13. The device of claim 2, wherein the multisink medium comprises a silicone-based paste or putty comprising ceramic particles.

14. The device of claim 2, further comprising an acoustic absorber layer attached to a side of the integrated circuit substrate opposite the one or more MEMs ultrasound transducers.

15. An imaging device comprising:
an integrated circuit substrate;
a first component coupled to the integrated circuit substrate with a gap being formed between opposing surfaces of the integrated circuit substrate and the first component;
a housing coupled to the integrated circuit substrate; and
a multisink medium comprising a silicone-based paste or putty containing ceramic particles, the multisink medium being thermally conductive and acoustically non-conductive and being flowed into the gap and conforming to opposing surfaces of the integrated circuit substrate and the first component to (i) facilitate thermal conductivity and (ii) reduce acoustic conductivity between the opposing surfaces, the multisink medium is in contact with the first component and reduces acoustic reflections reflecting off the first component.

16. The device of claim 12, wherein the multisink medium is disposed at least partially between one or more ultrasound transducers and an acoustically reflective material.

17. The device of claim 12, wherein the first component comprises a microelectromechanical (MEMs) ultrasound transducer.

18. The device of claim 12, wherein the first component comprises a housing.

19. An imaging device comprising:
one or more MEMS ultrasound transducers coupled to a circuit substrate;
a housing coupled to the circuit substrate to form a handheld casing for the imaging device, the housing defining an internal cavity between the housing and the one or more MEMS ultrasound transducers;
an acoustic absorber layer attached to a side of the circuit substrate opposite the one or more MEMs ultrasound transducers; and
a flowable multisink medium that is thermally conductive and acoustically non-conductive and is disposed within the internal cavity of the housing and filled in against irregularly shaped geometries, cavities, gaps, or spaces within the internal cavity, the multisink medium being in contact with the housing and the acoustic absorber layer and being configured to reduce acoustic imaging artifacts and facilitate heat transfer within the imaging device by filling the internal cavity for enhancing acoustic isolation of components opposite the transducers.

20. The device of claim 16, wherein the multisink medium completely fills the internal cavity.

21. The device of claim 16, wherein the multisink medium comprises an elastomer.
